(19) United States
(12) Patent Application Publication
(10) Pub. No.: US 2024/0108812 A1
(43) Pub. Date: Apr. 4, 2024

(54) SYRINGE CARRIERS

(71) Applicant: SHL MEDICAL AG, Zug (CH)

(72) Inventors: Daniel Säll, Segeltorp (SE); Daniel Carlsson, Enskede (SE); Slobodan Stefanov, Deerfield Beach, FL (US); Ming-Ting Yin, Taipei City (TW); Justin Stewart, Deerfield Beach, FL (US)

(21) Appl. No.: 18/275,336

(22) PCT Filed: Dec. 21, 2021
(86) PCT No.: PCT/EP2021/086937

§ 371 Date: Aug. 1, 2023

Related U.S. Application Data
(60) Provisional application No. 63/149,380, filed on Feb. 15, 2021.
(60) Provisional application No. 63/153,408, filed on Feb. 25, 2021.
(60) Provisional application No. 63/158,580, filed on Mar. 9, 2021.
(60) Provisional application No. 63/162,601, filed on Mar. 18, 2021.
(60) Provisional application No. 63/178,577, filed on Apr. 23, 2021.

(30) Foreign Application Priority Data
Jul. 23, 2021 (EP) 21187322.9

(51) Int. Cl. A61M 5/32; A61M 5/20

(52) U.S. Cl. CPC A61M 5/3204; A61M 5/20

(57) ABSTRACT

This application describes syringe carriers for medicament delivery devices such as autoinjectors. In particular, an example syringe carrier is tubular and extends along a longitudinal axis from a proximal end to a distal end, the syringe carrier comprising a base section at the distal end of the syringe carrier, the base section having a fixed circumference, at least one arm extending towards the proximal end of the syringe carrier from the base section, and an expandable ring attached to the proximal end of the at least one arm, wherein the ring extends entirely around the longitudinal axis of the syringe carrier in the circumferential direction. Various other syringe carriers and associated medicament delivery devices and methods are described.

### CROSS REFERENCE TO RELATED APPLICATIONS

The present application is a U.S. National Phase Application pursuant to 35 U.S.C. § 371 of International Application No. PCT/EP2021/086937 filed Dec. 21, 2021, which claims priority to European Patent Application No. 21187322.9, filed Jul. 23, 2021; U.S. provisional patent application 63/149,380, filed 15 Feb. 2021; U.S. provisional patent application 63/153,408, filed 25 Feb. 2021; U.S. provisional patent application 63/158,580, filed 9 Mar. 2021; U.S. provisional patent application 63/162,601, filed 18 Mar. 2021; and U.S. provisional patent application 63/178,577, filed 23 Apr. 2021. The entire disclosure contents of these applications are herewith incorporated by reference into the present application.

### TECHNICAL FIELD

Syringe carriers for medicament delivery devices such as autoinjectors.

### BACKGROUND

Medicament delivery devices such as autoinjectors often have a syringe carrier to support a syringe (typically a glass syringe) within the medicament delivery device. Although existing syringe carriers can be effective at supporting the syringe, the Applicants have appreciated that further improvements can be made.

### SUMMARY

The present disclosure concerns a number of different concepts for supporting a syringe in a medicament delivery device as described below.

The present disclosure is defined by the appended claims, to which reference should now be made.

An aspect concerns a syringe carrier for a medicament delivery device, wherein the syringe carrier is tubular and extends along a longitudinal axis from a proximal end to a distal end, the syringe carrier comprising a base section at the distal end of the syringe carrier, the base section having a fixed circumference, at least one arm extending towards the proximal end of the syringe carrier from the base section, and an expandable ring attached to the proximal end of the at least one arm, wherein the ring extends entirely around the longitudinal axis of the syringe carrier in the circumferential direction relative to the longitudinal axis. Provision of an expandable ring can allow the RNS of a syringe to expand the ring as the syringe is inserted into the syringe carrier (typically in the axial direction from the distal end). Provision of a ring can also be beneficial as it can reduce the tendency for syringe carriers to get tangled together when being transported in bulk or sorted during assembly. This can be particularly beneficial for use on automated assembly lines.

Optionally, the expandable ring is arranged to expand by breaking. Optionally, the expandable ring comprises a weak point at which the expandable ring is designed to break when a syringe is inserted into the syringe carrier.

Optionally, the expandable ring comprises one or more hinges spaced around the circumference of the expandable ring. Optionally, the expandable ring comprises a plurality of segments spaced around the circumference of the expandable ring and a plurality of hinges spaced around the circumference of the expandable ring. Optionally, each segment has a hinge on either side in the circumferential direction.

Optionally, at least one of the one or more hinges is a collet. Optionally, the collet extends entirely around the longitudinal axis of the syringe carrier in the circumferential direction.

Optionally, the expandable ring is circular in a cross-section perpendicular to the longitudinal axis. Optionally, the expandable ring is arranged to expand by increasing the diameter of the ring. An aspect concerns a syringe carrier for a medicament delivery device, wherein the syringe carrier is tubular and extends along a longitudinal axis from a proximal end to a distal end, the syringe carrier comprising a base section at the distal end of the syringe carrier, a connection section extending towards the proximal end of the syringe carrier from the base section, and a non-circular ring attached to the proximal end of the connection section, wherein the ring extends entirely around the longitudinal axis of the syringe carrier in the circumferential direction. Provision of a non-circular ring can allow the RNS of a syringe to deform the ring as the syringe is inserted into the syringe carrier (typically in the axial direction from the distal end). Provision of a ring can also be beneficial as it can reduce the tendency for syringe carriers to get tangled together when being transported in bulk or sorted during assembly. This can be particularly beneficial for use on automated assembly lines.

Optionally, the connection section is at least one flexible arm. Optionally, the connection section is a flexible sleeve. Optionally, the ring is oval. Optionally, the ring has a plurality of sections, and a first subset of the sections are further from the longitudinal axis than a second subset of the sections. Optionally, the ring has a plurality of sections, and at least one of the sections has a radius of curvature smaller than the average radius of curvature of the overall ring.

Optionally, the at least one arm extends in the longitudinal direction from a proximal end to a distal end. Optionally, the expandable ring is attached to the proximal end of each of the two arms. Optionally, the at least one arm is flexible. Optionally, the at least one arm comprises a slot extending in the longitudinal direction from the proximal end of the arm. Optionally, the at least one arm is attached to the part of the ring that is furthest from the longitudinal axis.

Optionally, the base extends entirely around the longitudinal axis of the syringe carrier in the circumferential direction. Optionally, the base is circular in a cross-section perpendicular to the longitudinal axis. Optionally, the proximal end of the syringe carrier is the end that would be closest to the medicament delivery member end when the syringe carrier is assembled in a medicament delivery device. Optionally, the ring is the proximal-most part of the syringe carrier. Optionally, the ring comprises a flange extending towards the longitudinal axis from the ring.

An aspect concerns a syringe carrier extending along a longitudinal axis from a proximal end to a distal end, the syringe carrier comprising: a support portion configured to support a shoulder of a syringe of a medicament delivery device, wherein the support portion comprises an expandable base extending around the longitudinal axis in a circumferential direction and a flange extending inwards from the base; and an attachment portion comprising an arm configured to secure the syringe carrier inside said medicament delivery device, wherein the arm is attached directly to the base. This can allow the RNS of a syringe to expand the base as the syringe is inserted into the syringe carrier (typically in the axial direction from the distal end). This structure can also be beneficial as it can reduce the tendency for syringe carriers to get tangled together when being transported in bulk or sorted during assembly. This can be particularly beneficial for use on automated assembly lines.

Optionally, the base is an expandable ring that extends entirely around the longitudinal axis of the syringe carrier in the circumferential direction. Optionally, the expandable ring comprises a plurality of segments around the circumference of the expandable ring and a plurality of hinges around the circumference of the expandable ring, and wherein each segment is attached to a hinge on either side in the circumferential direction. Optionally, the ring is circular.

Optionally, the base is an expandable c-clip. Optionally, the c-clip extends only part of the way around the longitudinal axis of the syringe carrier in the circumferential direction. The syringe carrier of claim 30, wherein the c-clip extends between 180 and 359 degrees, or preferably between 270 and 345 degrees, or most preferably between 300 and 330 degrees around the longitudinal axis of the syringe carrier in the circumferential direction.

Optionally, the arm is flexible. Optionally, the arm is attached to an outer surface of the base. Optionally, the arm extends in a distal direction from the support portion. Optionally, the arm comprises a surface facing towards the proximal end, the surface being configured to engage a distally facing surface of a medicament delivery device during medicament delivery device assembly. Optionally, the arm comprises a radially extending protrusion or recess, wherein the protrusion or recess is configured to engage a radially extending feature of a medicament delivery device during medicament delivery device assembly.

Optionally, the syringe carrier consists of the support portion and the attachment portion. Optionally, the arm is configured to engage a first attachment of said medicament delivery device during medicament delivery device assembly and a second attachment of said medicament delivery device in a completed medicament delivery device. Optionally, the ring has a fixed circumference.

An aspect concerns a syringe carrier, the syringe carrier comprising an expandable ring and an attachment portion, wherein the attachment portion is attached to a segment of the expandable ring, and wherein the attachment portion comprises an arm configured to engage a first medicament delivery device attachment point during assembly and to subsequently engage a second medicament delivery device attachment point in a completed medicament delivery device.

An aspect concerns a medicament delivery device sub-assembly comprising a housing, a syringe carrier according to any of claims 25 to 40 and a syringe, wherein the housing is tubular and extends from a proximal end to a distal end, wherein the housing comprises a first attachment point configured to engage the arm of the syringe carrier, wherein the housing comprises a second attachment point configured to engage the arm of the syringe carrier, and wherein the second attachment point is closer to the proximal end of the housing than the second attachment point.

Optionally, the first attachment point is a rim of a window of the housing. Optionally, the second attachment point is on an inner portion of the housing. Optionally, the first attachment point is configured to releasably engage the arm of the syringe carrier. Optionally, the second attachment point is configured to permanently engage the arm of the syringe carrier.

An aspect concerns a medicament delivery device comprising any syringe carrier as described above or any medicament delivery device sub-assembly described above. Optionally, the medicament delivery device is an autoinjector.

An aspect concerns a method of assembly of a medicament delivery device, the method comprising carrying out the following steps in the following order: providing a medicament delivery device housing, the housing extending from a proximal end to a distal end; inserting a syringe carrier according to one of claims 1 to 40 into the distal end of the housing and moving the syringe carrier in the proximal direction relative to the housing to a first position; inserting a syringe into the syringe carrier; and moving the syringe carrier and the syringe together in the proximal direction relative to the housing to a second position.

Optionally the syringe carrier surrounds a smaller area in a plane perpendicular to the longitudinal axis in the second position than in the first position. This can grip the syringe carrier in the second position (the syringe can enter the syringe carrier in the first position, and is then clamped in the second position so that the syringe cannot come back out of the syringe carrier).

An aspect concerns a method of assembling a medicament delivery device comprising the steps of attaching a syringe to a syringe carrier according to any of claims 1 to 40 and of subsequently inserting the syringe and the syringe carrier into a housing.

An aspect concerns a method of assembling a medicament delivery device, comprising the steps of providing a syringe carrier and subsequently inserting a syringe into the syringe carrier, wherein inserting the syringe into the syringe carrier breaks a ring of the syringe carrier.

### BRIEF DESCRIPTION OF THE DRAWINGS

Embodiments of the present disclosure will now be described by way of example only and with reference to the accompanying drawings as listed below.

FIG. 1 shows a cross-section view of part of an example autoinjector.

FIG. 2 shows a perspective view of an example syringe carrier.

FIG. 3 shows a perspective view of part of the syringe carrier of FIG. 2 during assembly.

FIG. 4 shows a perspective view of part of the syringe carrier of FIG. 2 during assembly.

FIG. 5 shows a close-up view of part of FIG. 1.

FIG. 6 shows a perspective view of another syringe carrier with a syringe for context.

FIG. 7 shows a perspective view of the syringe carrier of FIG. 6.

FIG. 8 shows a perspective view of another syringe carrier with a syringe for context.

FIG. 9 shows a perspective view of the syringe carrier of FIG. 8.

FIG. 10 shows a perspective view of another syringe carrier with a syringe for context.

FIG. 11 shows a perspective view of the syringe carrier of FIG. 10.

FIG. 12 shows a perspective view of another syringe carrier with a syringe for context.

FIG. 13 shows a perspective view of the syringe carrier of FIG. 12.

FIGS. 14 to 16 show perspective views of another example syringe carrier.

FIGS. 17 and 18 show partial cross-section views during assembly of an autoinjector comprising the syringe carrier of FIG. 14.

FIG. 19 shows a partial cross-section view of an autoinjector comprising the syringe carrier of FIG. 14.

FIG. 20 shows a cross-section view of an autoinjector comprising the syringe carrier of FIG. 14.

FIGS. 21 and 22 show perspective views of another example syringe carrier.

FIG. 23 shows a perspective view of an example housing.

FIG. 24 shows an end view of the example housing of FIG. 23.

FIGS. 25 and 26 show perspective views of another example syringe carrier.

FIGS. 27 and 28 show perspective views of another example syringe carrier.

FIGS. 29 and 30 show perspective views of another example syringe carrier.

FIGS. 31, 32 and 33 show perspective views of three more example syringe carriers.

FIG. 34 shows a perspective view of another example syringe carrier.

FIGS. 35 and 36 show partial cross-section views of the syringe carrier of FIG. 34 in a medicament delivery device at different points of medicament delivery device assembly.

FIG. 37 shows a partial cross-section view of the syringe carrier in a medicament delivery device.

FIG. 38 shows a perspective view of another example syringe carrier.

FIG. 39 shows a perspective view of the syringe carrier of FIG. 38 attached to a syringe.

FIG. 40 shows a perspective view of another example syringe carrier.

FIG. 41 shows a partial cross-section view of the syringe carrier of FIG. 34 in a medicament delivery device during medicament delivery device assembly.

FIG. 42 shows a partial cross-section view of the syringe carrier in a medicament delivery device.

FIG. 43 shows a perspective cross-section view of part of the housing of the medicament delivery device of FIG. 42.

FIG. 44 shows a perspective view of an example autoinjector.

FIG. 45 shows a perspective view of an example syringe.

FIG. 46 shows a cross-section view of the example syringe of FIG. 45.

### DETAILED DESCRIPTION

Embodiments of the present disclosure will now be described by way of example only and with reference to the accompanying drawings. The present disclosure is defined by the appended claims, to which reference should now be made.

In general, the present disclosure describes various syringe carriers for medicament delivery devices such as autoinjectors. Generally, these syringe carriers are for use in devices in which the syringe is inserted into the device (for example into a housing of the device) in the axial direction during assembly.

The ideas that will now be described focus on various syringe carriers with an expandable front ring.

As context, it is noted that, when using a prefilled syringe inside an autoinjector, the syringe needs to be supported in a safe way. In some cases a “finger-flange” at the distal end of the syringe is used as support, but this could lead to cracks in the glass syringes in some cases. The neck of the syringe provides a safer support but if the syringe is equipped with an RNS (rigid needle shield), as with a typical 1 ml syringe, the RNS has the same diameter as the syringe. This can make it difficult to grip around the neck without having some type of snap feature. A C-clip shape (C-shape), for example, could be used, but can provide an uneven support of the syringe, meaning that the syringe needle might be dislocated from the centre when the plunger rod force is applied. In case of manual insertion devices, this means the needle can move sideways inside the body when the device is activated, which can cause pain. Another potential problem with C-clip shapes is that in automated assembly the C-clip shaped parts have a tendency to tangle and cause problems.

Another solution is “bendable fingers”. However, it is very important to be able to see the syringe to inspect the drug and the movement of the plunger. There is typically too short a distance available for finger flanges in the area of the window, meaning that it is difficult to get an even 360-degree support of the syringe.

Considering the potential problems with the existing designs, the applicant has appreciated that alternative approaches could provide a more satisfactory solution.

The syringe carriers of the present disclosure which will now be described integrate an expandable ring into the syringe carrier. This can provide an even syringe support (that is, the syringe is supported evenly around its circumference), which can minimise or avoid the risk of sideways movements when force is applied to the syringe. Another positive effect is that tangling problems during feeding in automated assembly lines can be improved compared to C-shapes. The expandable ring can also be short in the longitudinal direction which can enable a large inspection window for the drug.

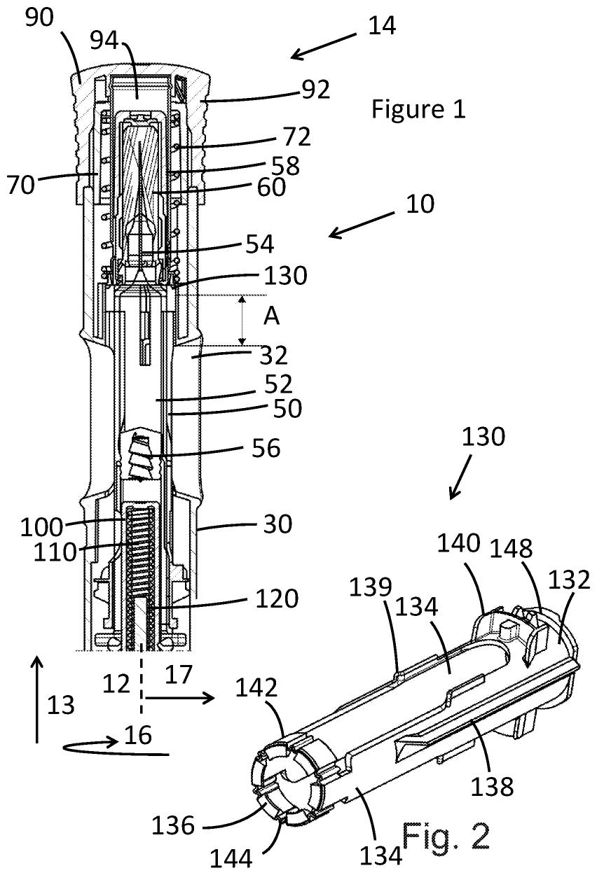

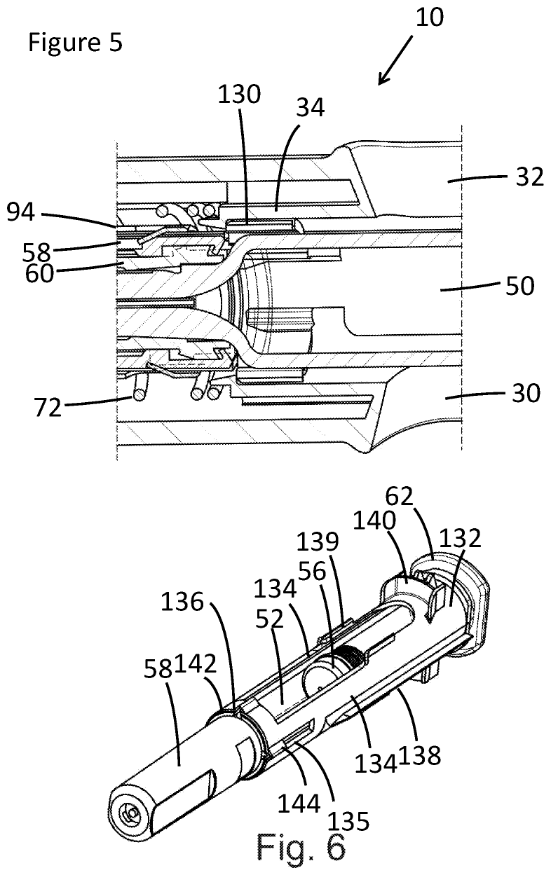

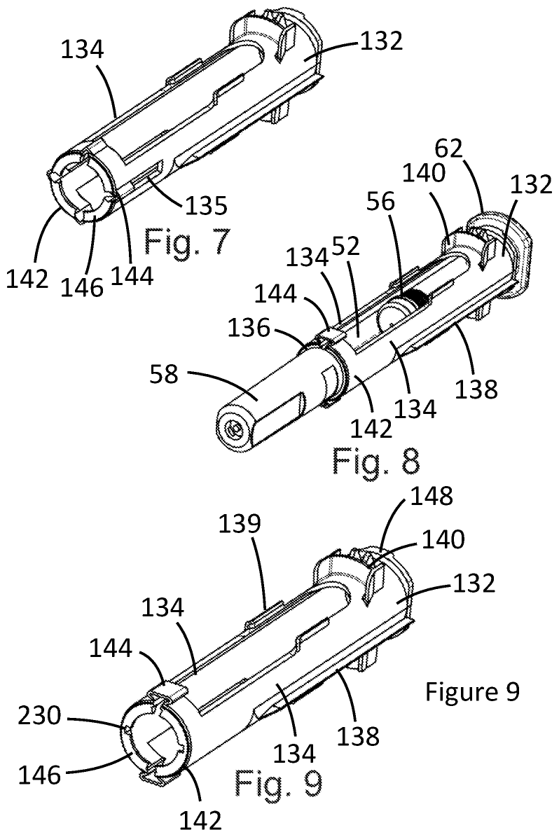

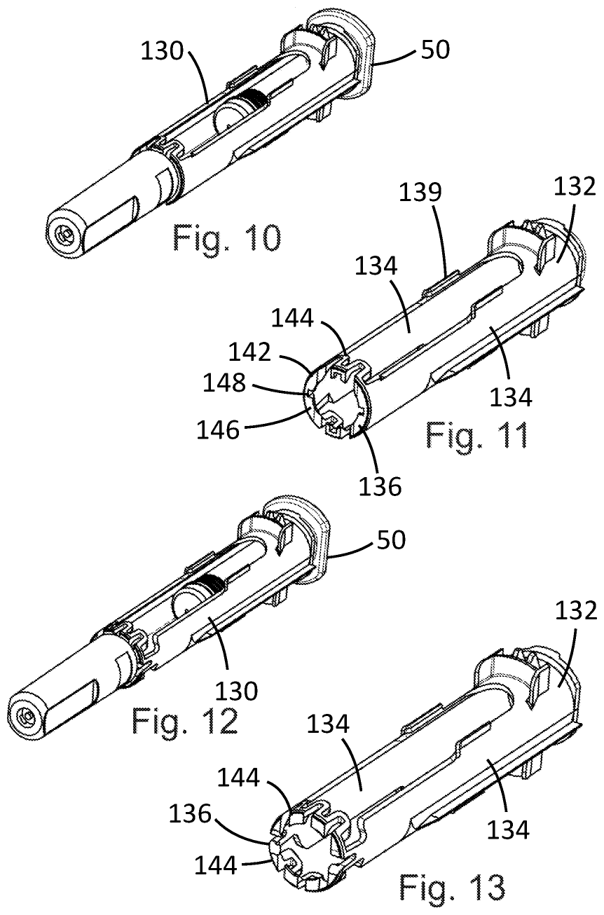

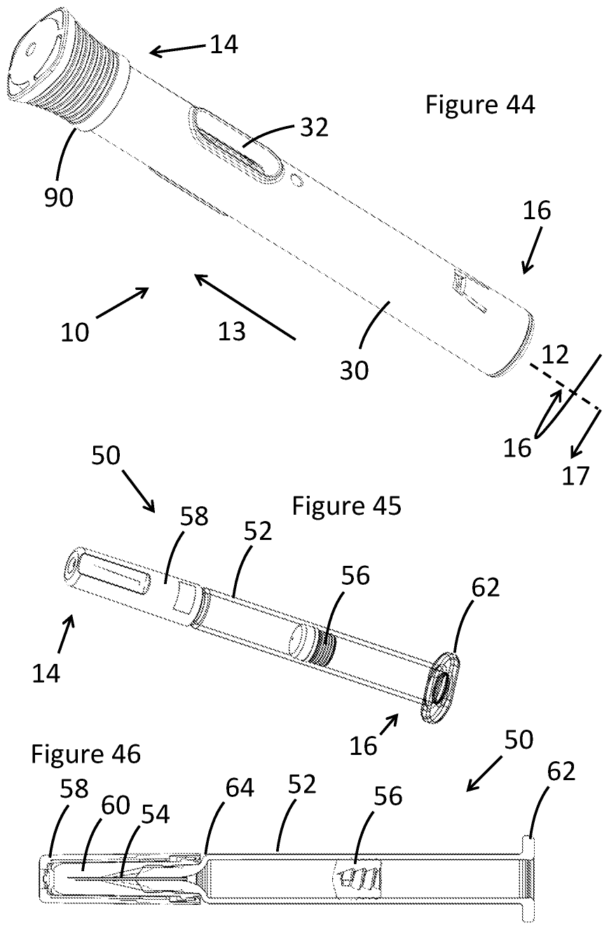

FIGS. 1 to 5 show an example syringe carrier, with FIGS. 1 and 5 showing the syringe carrier in the context of an example autoinjector, and FIGS. 2 to 4 showing the syringe carrier in further detail. FIGS. 6 and 7 show an example syringe carrier with 4 segments rather than the 6 segments shown in FIGS. 2 to 4. FIGS. 8 and 9 show an example syringe carrier with two segments. FIGS. 10 and 11 show an example syringe carrier with a collet design, in this case with part of the ring as a collet. FIGS. 12 and 13 show another example syringe carrier with a collet design, this time with the full circumference of the ring as a collet. It is noted that the other parts of the syringe can also be varied, and that the particular structure shown in the examples is not essential. FIG. 44 (along with FIG. 1 in particular, and also FIG. 5) shows an example of an autoinjector that could use the syringe carrier designs described above, although the syringe carrier designs described above could also be used with other autoinjectors and other medicament delivery devices, and are not limited to this one particular design of autoinjector. The same is true for the other ideas herein.

FIG. 1 shows an example autoinjector extending along an axis 12 in an axial direction between distal end (not shown) and a proximal end 14, with a radial direction 17 and a circumferential direction 16 also depicted for reference. The autoinjector comprises a housing (or body) 30, a syringe carrier (syringe holder) 130, a syringe 50, an optional needle guard 70, an optional needle guard spring 72, an optional cap 90, and elements of a powerpack, including in this example a plunger rod 100, a plunger rod spring 110 and a guide rod 120. The housing 30 comprises a window 32. The syringe 50 comprises a medicament holder 52 (medicament barrel), a needle 54, a stopper 56 and a needle shield, in this case comprising a flexible needle shield 60 and a rigid needle shield (RNS) 58. The cap 90 comprises a cap housing 92 and a rigid needle shield (RNS) remover 94.

Preferably, the distance A (which is the distance in the axial direction between the proximal end of the window of the housing and the proximal end of the medicament container of the syringe) is kept relatively short so that at least part of the plunger rod remains visible after injection completion.

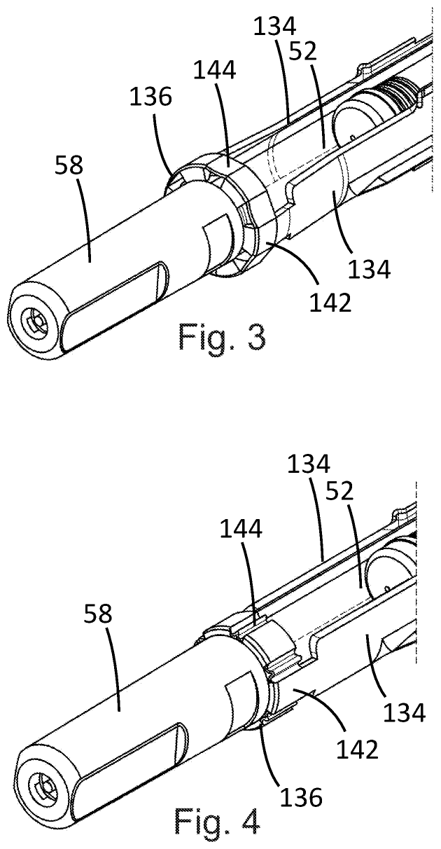

FIG. 2 shows an example of a syringe carrier 130. The syringe carrier 130 comprises a base 132 (base section), two arms 134 extending in the axial direction from the base 132, and an expandable ring 136. The expandable ring 136 is attached to the proximal end of the arms 134. The syringe carrier 130 also comprises several optional protrusions and ribs that can help align and/or help with medicament delivery device assembly, such as ribs 138, 139, a protrusion 140 and a distal flange 148. The ring 136 comprises six segments 142 spaced around the circumference of the ring and six expandable hinges 144 spaced around the circumference of the ring, with each segment 142 having a hinge 144 on either side in the circumferential direction. As can be seen from FIG. 3 in particular, each arm 134 is attached to a single segment 142 (although attachment of an arm to multiple segments is also possible, as shown in FIG. 7, for example).

FIGS. 3 and 4 show the syringe carrier 130 of FIG. 2 in the context of a syringe 50. As the syringe 50 is pushed in the proximal direction relative to the syringe carrier 130 during assembly, the ring 136 expands to allow the rigid needle shield 58 of the syringe 50 through the ring 136, as shown in FIG. 3. The arms 134 (or the proximal portion of the arms 134) are preferably flexible to allow the arms to move apart and help accommodate the expansion of the ring, though this is not essential depending on the structure of the ring. Once the rigid needle shield 58 has passed the ring 136, the ring can contract again. Practically speaking, this means that the inner diameter of the ring is less than the diameter of the corresponding RNS, and during device assembly can expand to a diameter equal to or greater than the corresponding RNS and subsequently back to a diameter less than the diameter of the corresponding RNS.

FIG. 5 shows a cross-section of part of the autoinjector of FIG. 1. When mounted, another part of the autoinjector prevents the ring from expanding, although this is not essential—for example the ring 136 can alternatively (or additionally) be biased to remain in the unexpanded position (the position shown in FIG. 4), for example by biasing the hinges and/or the arms of the syringe carrier. In this example, the housing prevents the ring 136 of the syringe carrier from expanding, although another autoinjector part could also be used. In this example, a ring 34 (inner part) of the housing 30 prevents the ring 136 of the syringe carrier from expanding, although this could alternatively be a protrusion, arm, rib or wall of the housing. In this example, the needle guard spring 72 is also supported at its distal end by the ring 136, though this is also optional and another part of the housing or of the autoinjector could support the needle guard spring 72.

FIG. 6 shows another example syringe carrier with a syringe for context. FIG. 7 also shows the syringe carrier. The syringe comprises a medicament holder 52, a stopper 56, an RNS 58 and a flange 62. The syringe carrier comprises a base 132 (base section), two arms 134, a ring 136, optional ribs 138, 139 and optional protrusions 140. The ring comprises four segments 142 and four hinges 144, arranged in the same way as described above for the syringe carrier of FIG. 2. In contrast to the syringe carrier of FIG. 2, the syringe carrier of FIG. 6 has each arm attached to two segments of the ring. The proximal end of each arm comprises a slot extending from the proximal end of the arm; this slot 135 allows the adjacent hinge to expand and contract. The slot is optional in some examples, but is provided in this example as there is a hinge aligned in the longitudinal direction with an arm.

FIGS. 8 and 9 show another example syringe carrier. The design is similar to those described above with FIGS. 2 and 6, and a full description of the syringe carrier will therefore not be repeated.

Optionally, one or more of the segments 142 comprises a flange 146 extending towards the axis. The flange can help align the syringe carrier and the syringe by slotting into the gap between the medicament holder 52 and the RNS 58 (specifically between the shoulder 64 and the RNS 58). Such optional flanges can also be seen on the other example syringe carriers. Optionally, one or more of the flanges 146 comprises a notch 230 to improve flexibility of the flange during device assembly.

FIGS. 10 and 11 show another example syringe carrier, and FIGS. 12 and 13 show yet another example syringe carrier. The designs are similar to those described above with FIGS. 2, 6 and 8, and a full description of the syringe carrier will therefore not be repeated. In these examples, the hinges are concertina shaped—instead of a single flexible arc of material, multiple arcs are combined, typically resulting in a sinusoidal—or zigzag-shaped hinge. In the examples in FIGS. 10 and 12, the concertina shape is angled so that the concertina is in the circumferential plane of the ring (that is, the concertina is angled so that the concertina describes its shape in a circumferential plane of the ring), but the concertina shape could alternatively be angled in a different direction, for example with the concertina extending in a plane perpendicular to the axis (so that the concertina describes its shape in a plane perpendicular to the axis). To put it another way, with a concertina that describes its shape in a circumferential plane of the ring, the length of each individual section of the concertina in the axial direction varies as the concertina moves from a contracted state to an expanded state (and the length in the radial direction remains the same). With a concertina that describes its shape in a plane perpendicular to the axis, the length of each individual section of the concertina in the radial direction varies as the concertina moves from a contracted state to an expanded state (and the length in the axial direction remains the same). The concertina shape could be considered to be a collet, with FIG. 13 showing an example with a collet structure around the entire circumference and FIG. 11 showing an example with collet sections interspersed with segments.

The hinges shown in FIG. 10 are still interspersed with segments as described in the examples above, but in the example of FIG. 12, no segments are provided, and a hinge (or in this case two hinges) extend the entire way around the ring. The ring therefore consists of two (or more generally one or more) hinges.

In general, the expansion of the ring is provided by the hinges and the segments in between the hinges do not need to change in shape. However, it can also be beneficial for the segments to be flexible as well, to reduce stresses within the ring during expansion and contraction of the ring.

The ideas that will now be described focus on syringe carriers with a non-circular front ring.

The issues here are similar to those described above, but will be described for completeness. The 1 ml tall syringe is by far the most used primary container within the auto injector market. The syringe usually is made out of glass; a material less suited for impact loads. The 1 ml tall syringe can be fitted with a flexible needle shield only or with an additional rigid needle shield. The latter can protect the user from needle stick injuries when administered as a prefilled syringe only, but can create a problem when assembled into an auto injector. When the diameter of the RNS is equally large as the barrel diameter of the 1 ml tall syringe, this limits the possibility of supporting the syringe at its neck or shoulder. Supporting the syringe by the finger flange is a relatively risky way to go since the flange is more sensitive to breakage due to its geometrical shape. This problem is usually solved by having a c-shaped support that is allowed to bend during syringe assembly or by flexing supports that are later supported by the auto injector enclosure. However, splitting the syringe carrier means the parts can tangle during bulk transport and the shape can easily be deformed prior to assembly if subjected to forces during transport.

Considering the potential problems with the existing designs, the applicant has appreciated that alternative approaches could provide a more satisfactory solution.

By designing the support front of the syringe carrier with a non-circular shape, like an oval for example, the front (proximal end of the syringe carrier) is closed and prevented from tangling, but allowed to flex into a circular shape during assembly allowing the RNS to pass. To prevent the front from deforming in a similar manner during usage, the body (housing) can be used to block this movement.

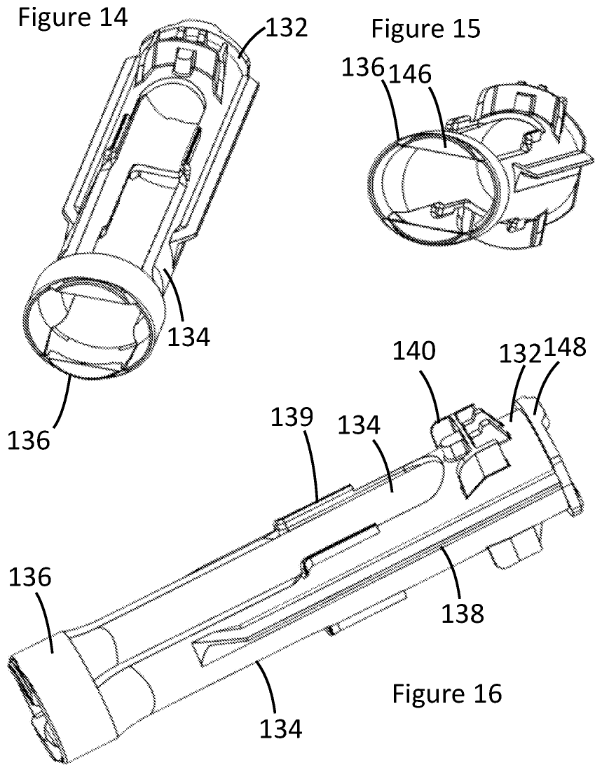

FIGS. 14, 15 and 16 show different angles of an example syringe carrier with an oval ring (front ring). Various other shapes, such as an n-sided regular shape where n is 3 or more (for example 4 or more, 5 or more, between 3 and 10, or between 4 and 6) such as a triangle, a square, a diamond, or a hexagon could also be used, as could an irregular shape. The arms are attached to the part of the ring that is furthest from the axis, although the arms could alternatively be attached to another part of the ring. The arms would generally be flexible to allow the ring to flex more easily. Other than the provision of a non-circular ring rather than an expanding ring, the syringe carrier design is similar to those described above (for example FIG. 2) and a description of the rest of the syringe carrier will therefore not be repeated in full.

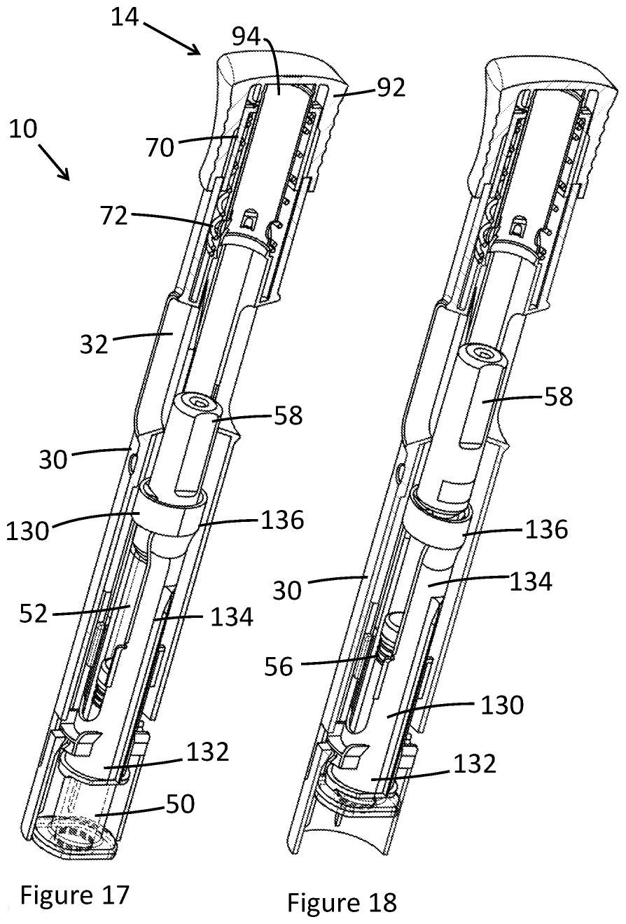

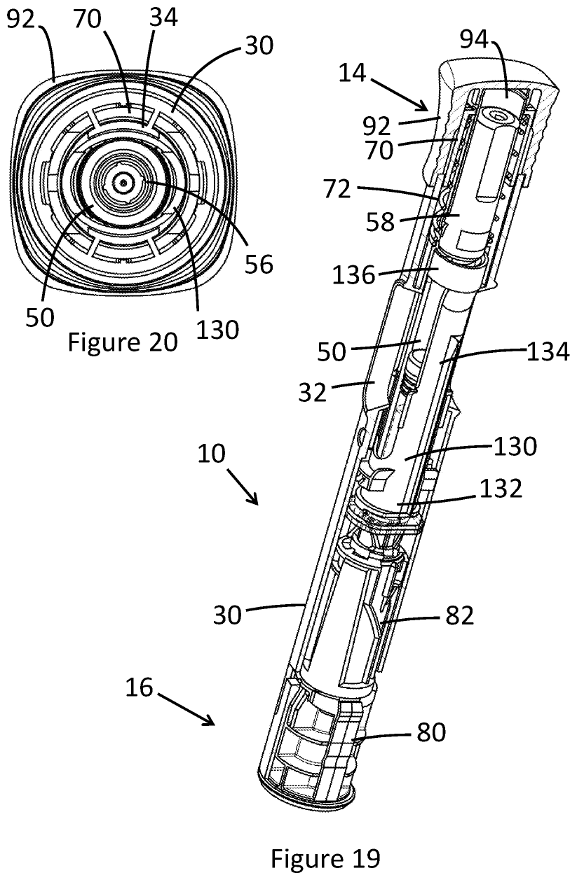

FIG. 17 shows a view of how the front of the syringe carrier flexes to allow the RNS to pass the syringe support during syringe assembly. FIG. 18 shows a view of how the front of the syringe carrier returns to its original shape and supports the neck of the syringe after the RNS has passed the oval shaped front. FIG. 19 shows a view of how the syringe carrier has reached its seat in the enclosure (inside the housing). The oval shape is blocked from expanding by the seat of the enclosure body (for example by an inner part of the housing). This particular supporting approach is not essential, but can help with stability of a completed medicament delivery device. In FIG. 19, an entire medicament delivery device is shown, including an example powerpack comprising a rear housing 80 and a rotator 82, a cap comprising a cap housing 92 and a rigid needle shield (RNS) remover 94, a housing 30 including a window 32, a syringe 50, the syringe carrier 130, a needle guard 70 and a needle guard spring 72. The other syringe carriers described herein could also be used in such a device. FIG. 20 shows a cross sectional view of the final assembly with the oval shaped front supported by the inner geometry of the enclosure (the flanges 146 are not visible due to the placement of the cross-section). In this example, an inner part 34 of the housing 30 is adjacent to the ring 136 of the syringe carrier 130 (specifically-adjacent to the part of the ring 136 that is closest to the axis), thereby stopping the ring 136 from becoming round and thereby releasing the syringe by disengaging the flange 146 from the syringe. In this case, the inner part 34 of the housing has a circular inner diameter, and could have the same structure as the inner part 34 of the housing shown in FIG. 5 (and can also have other functions, such as supporting the needle guard spring 72 as shown in FIG. 5). In general, though, provision of a support such as the inner part 34 of the housing (actually two separate supports opposite one another relative to the axis, in this particular example) is optional. Differently shaped supports (either as another part of the housing 30 or as part of another component) could alternatively be provided.

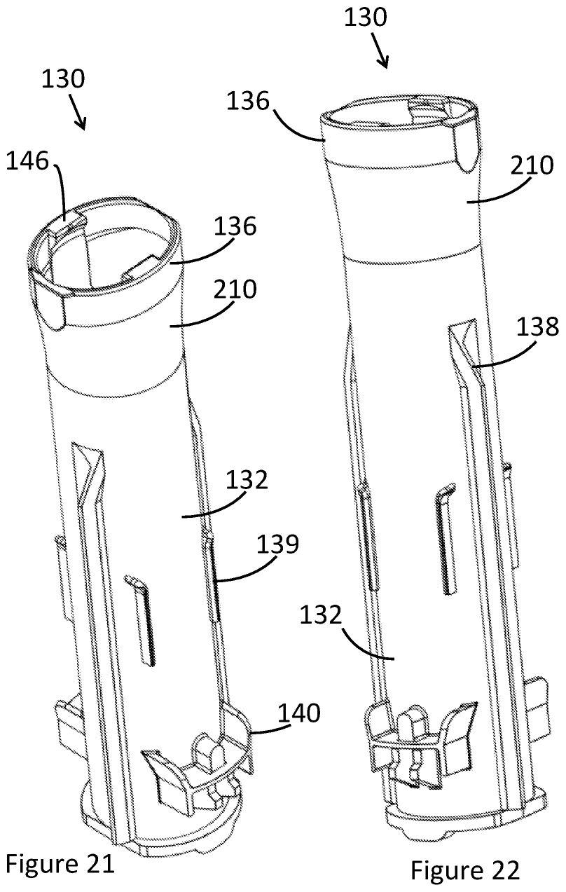

FIGS. 21 and 22 show another similar syringe carrier. The syringe carrier 130 comprises a ring 136, in this case an oval ring. The ring is once again flexible. Instead of arms, the ring 136 is attached to the base 132 (base section) of the syringe carrier 130 by a sleeve 210. The sleeve is flexible to allow the ring to flex. As with the other examples shown above, the ring may comprise a flange 146 extending towards the longitudinal axis from the ring. The base 132 is tubular. The base may be partially or entirely made from a transparent material to allow the syringe inside the syringe carrier to be seen by a user of a completed medicament delivery device, despite the syringe carrier being in the way.

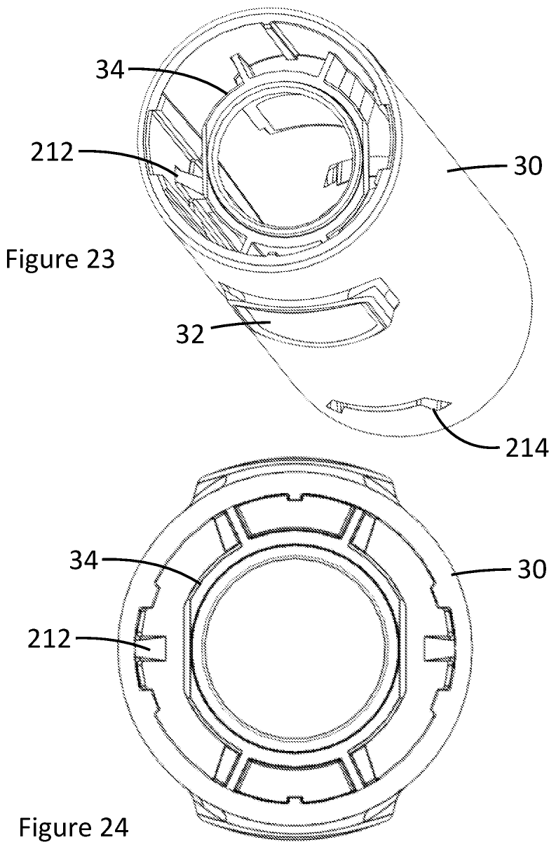

FIGS. 23 and 24 show an example housing 30; this housing could be used with the syringe carrier in FIGS. 21 and 22 or more generally with the syringe carriers and medicament delivery devices described elsewhere in this application. In this example, the inner part 34 of the housing is a ring instead of two separate segments as shown in FIG. 19. Various other optional housing features are shown, such as a protrusion 212 and a cut-out 214, which can help attach the housing to other medicament delivery device components.

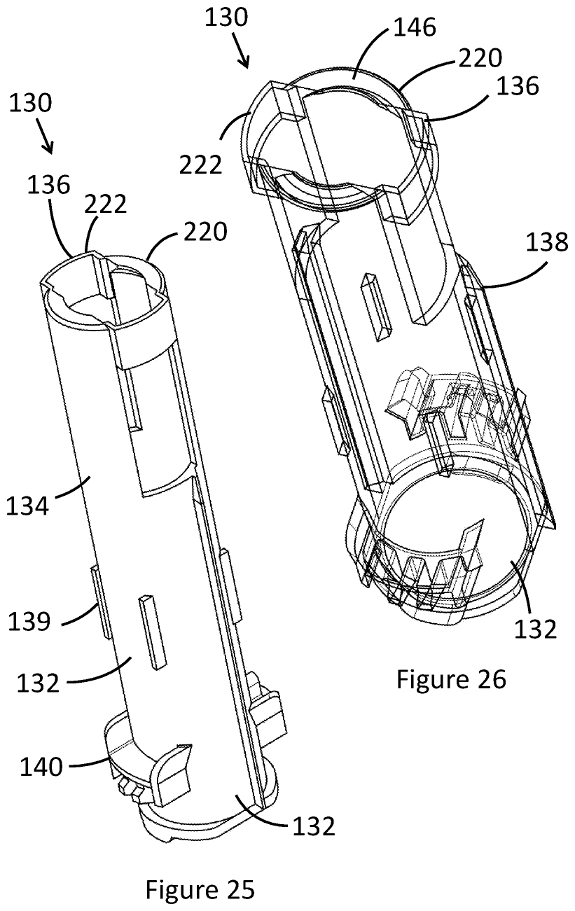

Another syringe carrier is shown in FIGS. 25 and 26. The ring at the proximal end of the syringe carrier is non-circular, similar to those above in FIGS. 14 to 24. This allows the proximal end of the syringe carrier to flex to allow an RNS of a syringe to pass, and then to flex back to hold the syringe once the RNS has passed. Rather than an oval shape, a first subset of the segments of the ring (inner segments 220 of the ring) are further away from the axis than a second subset of the segments of the ring (outer segments 222 of the ring). In this example, each of the first subset and the second subset has two segments, and the segments alternate (inner-outer-inner-outer) around the circumference of the ring, but other structures and other numbers of segments could be used. As a minimum, at least one inner segment and at least one outer segment are needed.

When a syringe is pushed through the ring, the segments closer to the axis are pushed away from the axis, and the segments further from the axis are pushed towards the axis. In this example, the flanges 146 are attached to the segments of the ring that are closer to the axis, and the segments of the ring that are closer to the axis are attached to the arms 134 whereas the segments of the ring that are further from the axis are not attached to the arms, though this particular structure could also be varied.

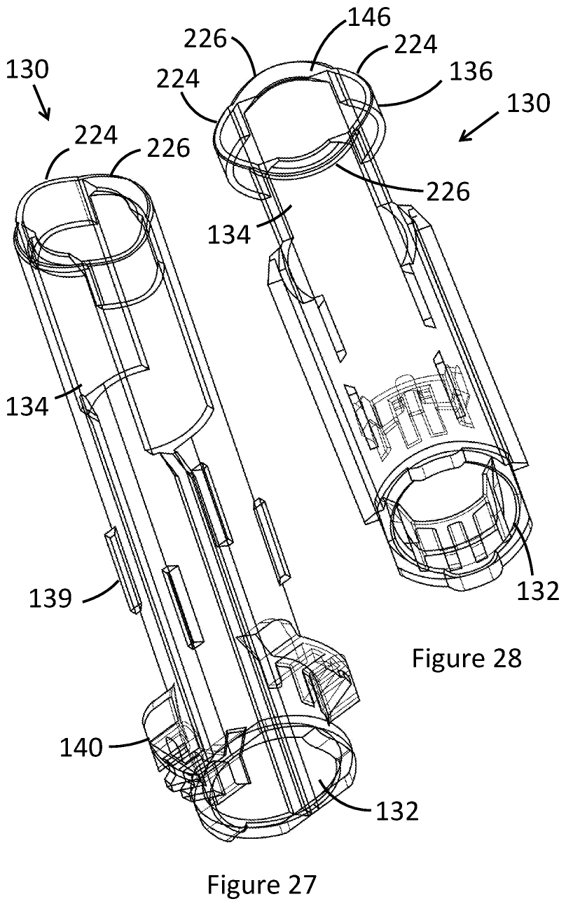

Another syringe carrier is shown in FIGS. 27 and 28. The ring at the proximal end of the syringe carrier is non-circular, similar to the ideas in FIGS. 14 to 26. This allows the proximal end of the syringe carrier to flex to allow an RNS of a syringe to pass, and then to flex back to hold the syringe once the RNS has passed. In this example, some of the segments of the ring have a smaller radius of curvature than the average radius of curvature of the overall ring (these are reduced-radius-of-curvature segments 224 of the ring 136; for contrast, the other segments are marked as normal-radius-of-curvature segments 226 of the ring 136). This allows these segments 224 to flex and thereby increase their radius of curvature when pushed outwards by an RNS, allowing the radius of the ring as a whole to increase to let the RNS pass.

The exact radius of curvature needed for the segments of the ring with a smaller radius of curvature than the ring would depend on the amount that the ring needs to be able to expand to let the syringe through. In this example, two reduced-radius segments 224 and two normal segments 226 are provided, with the segments alternating (i.e. reduced radius-normal radius-reduced radius-normal radius) around the circumference of the ring, but other structures and other numbers of segments could be used. At a minimum, two segments are needed, with a minimum of one reduced-radius segment. Some or all of the segments could be reduced-radius segments.

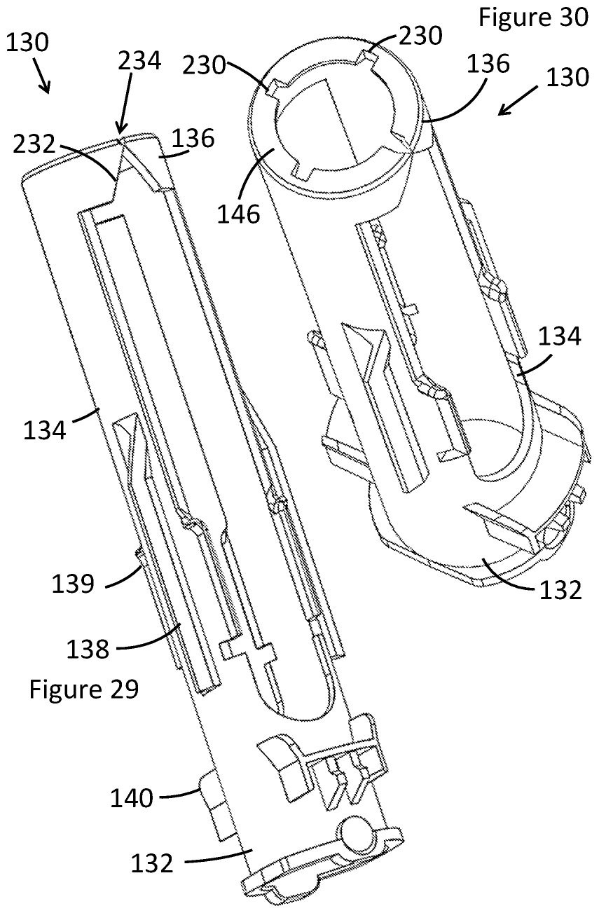

Another syringe carrier is shown in FIGS. 29 and 30. In this example, the proximal end of the syringe carrier comprises an expandable ring that extends all the way around the axis (or alternatively almost all the way around, except for a small gap). A slit or discontinuity (or alternatively more than one) in the ring in the circumferential direction helps the ring of the syringe carrier to flex to allow an RNS of a syringe to pass, and then to flex back to hold the syringe once the RNS has passed. In this particular example, a V-shaped slot 232 is provided on the distal side of the proximal end and a second (in this case smaller) V-shaped slot 234 is provided on the proximal side of the proximal end, with the two sides of the ring touching (but not directly attached to one another, so as to allow flexing) when in a relaxed state. The V-shaped nature of the slot is not essential, though, and other shapes could be used for the slot, including with both sides of the slot parallel to one another (and either touching or slightly spaced apart) and either extending parallel to the longitudinal axis or at an angle relative to the longitudinal axis. Provision of no gap (rather than slightly spaced apart) could be beneficial as it can reduce the possibility of syringe carrier components getting tangled during bulk component transport.

Several optional notches 230 are provided in the flange 146 to make it easier for the ring to flex. When the ring flexes, the ring preferably remains circular or generally circular, as a flex in this manner provides less stress on the syringe carrier than if the flexing occurred only at a single point in the ring. There may be more flexing at the points around the ring where a notch 230 is present.

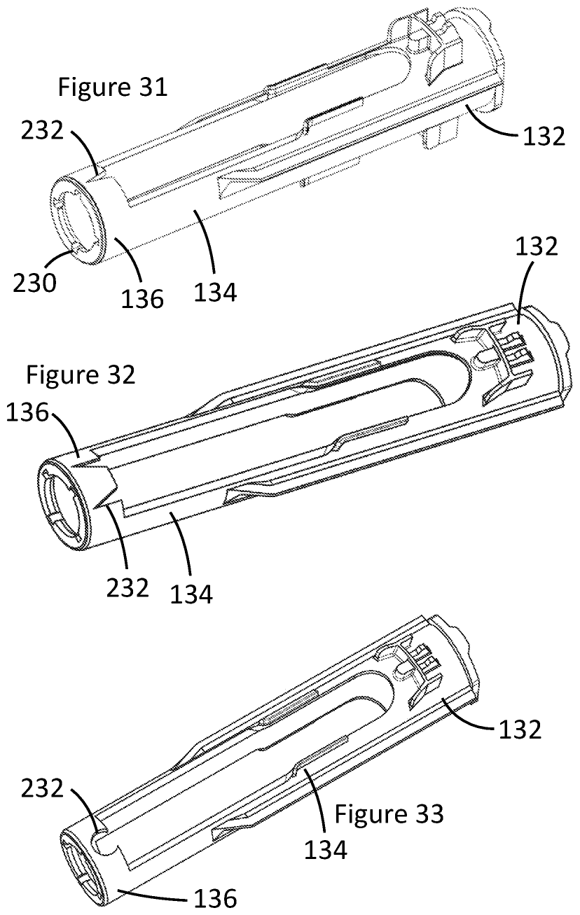

Another syringe carrier is shown in FIG. 31. This basic structure is similar to the examples described with respect to FIGS. 29 and 30 and the full description will not be repeated for succinctness. In this example, the proximal end of the syringe carrier comprises a breakable ring that extends all the way around the axis, instead of an expandable ring as in FIGS. 29 and 30. That is, the ring only expands by being broken, rather than due to a gap in the ring. Two alternative examples are shown in FIGS. 32 and 33. In each example, at least one slot 232 is provided in the ring; the slot extends part of the way through the ring in the longitudinal direction. During assembly of a medicament delivery device, a syringe would be pushed through the ring (to a final position as shown in FIG. 4, for example), and pushing the RNS of the syringe through the ring breaks the ring at the slot (since the slot provides a weak point in the ring). The ring can then expand to allow the RNS to pass.

In FIG. 31, a V-shaped slot is provided on the distal side of the proximal end. The V-shaped nature of the slot is not essential, though, and other shapes could be used for the slot (as for example in FIG. 33), including with both sides of the slot parallel to one another and either extending parallel to the longitudinal axis or at an angle relative to the longitudinal axis. The slot could extend from the proximal end of the ring rather than the distal end. Where two or more slots are provided (such as in FIG. 32), the slots could all extend from the distal end of the ring, all extend from the proximal end of the ring, or some could extend from the proximal end of the ring and some from the distal end of the ring. Potentially relevant factors in varying the geometry of the ring and the slot could include the shape of the syringe, the strength of the RNS of the syringe, and the desired force needed to break the ring.

This approach can be beneficial compared to syringe carriers with a c-clip shaped proximal end, as having the ring at the proximal end extending a full 360 degrees around the circumference can reduce or eliminate problems with syringe carriers getting tangled up together during bulk transport of syringe carriers. Removal of the gap provided in this idea can make it even harder for tangling to occur. Due to the breakage of the syringe carrier ring, this approach can also provide a visible, audible and/or tactile indication that assembly is proceeding as expected.

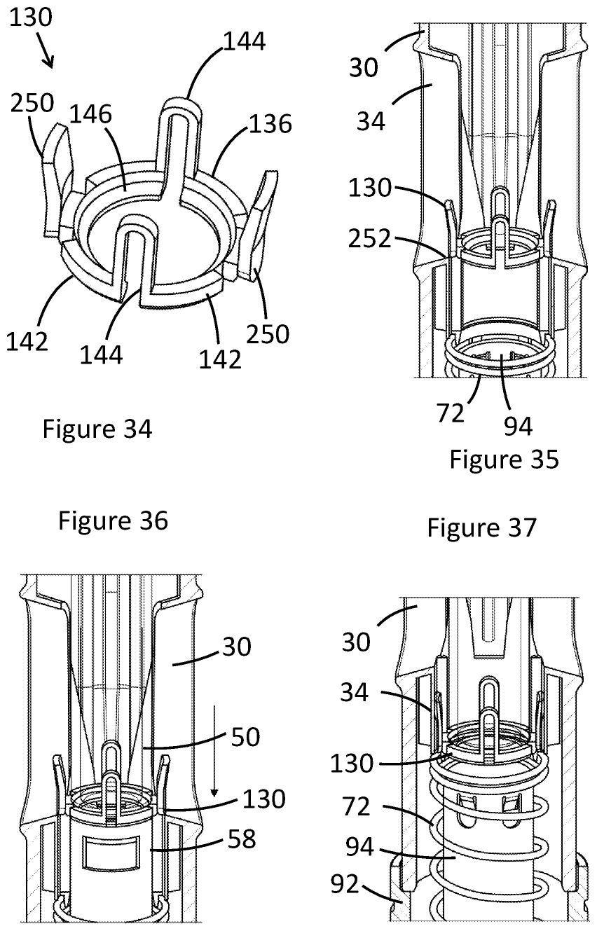

FIGS. 34 to 37 show another idea. In general, the cylindrical body of the syringe housing (syringe carrier) which surrounds the syringe barrel (medicament holder) is removed. The remaining part is a collar restricting proximal motion of the syringe in final assembly and also providing several structures for assembly process.

The syringe carrier, as shown in FIG. 34 in particular, comprises an expandable ring (a base). The expandable ring comprises two halves, with the two halves attached to one another by two clip springs. A side plate is attached to each of the two halves—the two side plates make up an attachment portion. Considering the example of FIG. 34 in more detail, the syringe carrier 130 comprises a ring 136 (a base) comprising two segments 142 and two hinges 144. A flange 146 extends towards the longitudinal axis from each of the segments 142. An arm 250 is attached to each of the segments 142. The arms each extend in the longitudinal direction from a proximal end to a distal end, and the proximal end of each of the arms is attached to their respective segment 142. The arms would generally be flexible so that the proximal end of the arm can move (in the radial direction in this example), though the arms could alternatively be rigid, and the features of the medicament delivery device (for example of the housing of the medicament delivery device) with which they interact could be flexible.

During assembly, the syringe carrier is inserted into the distal end of the housing and moved in the proximal direction relative to the housing until the side plates are temporarily clamped on the edge (rim 252) (in this case the edge closest to the proximal end of the housing) of the windows 32 of the housing 30 of a medicament delivery device as shown in FIG. 35 (or on another structure such as protrusions extending from an inner surface of the housing or of another medicament delivery device component). In other words, a proximally facing surface on the syringe carrier (in this example on the proximal end of the arms 250, though it could be elsewhere on the arms or elsewhere on the syringe carrier) abuts a distally facing surface of the medicament delivery device (in this example a distally facing surface of the rim of the window of the housing 30).

When the syringe 50 is inserted into the housing (or body), the RNS 58 can be pushed through the clip (syringe carrier 130), as shown in FIG. 36. The hinges 144 expand to allow the RNS 58 through the ring 136, before contracting again (either by springing back into place or being pushed back into place). The syringe is then in its final position relative to the syringe carrier, but still needs to move further in the proximal direction relative to the housing 30. As the syringe 50 subsequently continues moving in the proximal direction into the housing during assembly, the syringe carrier 130, which is now attached to the syringe 50 between the RNS 58 and the shoulder 64 (by radial projections of the ring 136, specifically by a collar (flanges 146) of the ring in this example), is therefore pulled in the proximal direction along with the syringe 50. This step compresses the side plates (arms 250), pushing them towards the axis and unclipping them from the window and allowing the syringe and syringe carrier to move in the proximal direction to a final position in which the syringe carrier is fixed relative to the housing and the syringe, as seen in FIG. 37 (movement from a first attachment point to a second attachment point, wherein the second attachment point is closer to the proximal end of the housing than the first attachment point). In this example, the syringe carrier engages an inner part 34 of the housing 30 to secure the syringe carrier in the housing, although the syringe carrier could engage another part of the housing or another medicament delivery device component. The syringe carrier could be attached to the housing by various methods, for example a friction fit or a snap fit. As the inner housing surrounds the syringe carrier, the position of the inner housing can restrict expansion of the syringe carrier, thereby helping ensure that the syringe cannot fall out of the syringe carrier in a completed medicament delivery device.

For context, a number of other example medicament delivery device features are shown in FIGS. 35 to 37; these will not be described in detail as they have already been described in other parts of the application, for example with reference to FIGS. 1 and 44.

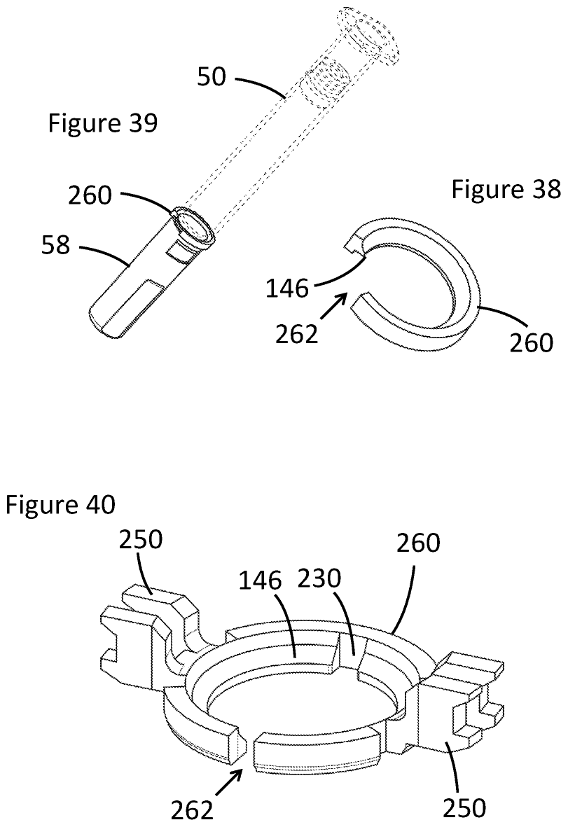

Another alternative is a syringe carrier that is c-clip shaped. An example of this is shown in FIGS. 38 and 39. The syringe carrier comprises a c-clip 260 with a flange 146 as shown in FIG. 38. The syringe carrier is shown attached to a syringe in FIG. 39, with part of the syringe shown as see-through (dashed lines) to better show the location of the syringe carrier. The syringe carrier is attached to the syringe from the side (radial direction) by pushing the neck of the syringe towards the notch (or slot) 262 of the c-clip and is subsequently assembled into a medicament delivery device by placing the syringe in the medicament delivery device, for example by inserting the syringe into a distal end of a housing of a medicament delivery device. The c-clip of the syringe carrier can then support the syringe from the neck by a collar that extends into the gap between the RNS and the medicament holder, and the syringe carrier itself can interact with another part or parts of the medicament delivery device, for example with the housing, to hold the syringe in place relative to other parts of the medicament delivery device.

A second example of a c-clip shaped syringe carrier is shown in FIGS. 40 to 43. As shown in FIG. 40, the structure of the syringe carrier is similar to the syringe carrier shown in FIG. 34, but with the ring replaced with a c-clip. The arms are also a somewhat different structure, but the arms in the examples in FIGS. 34 and 40 are interchangeable. In FIG. 40, the c-clip does not extend fully around the longitudinal axis, with a gap left by a notch 262 (though the c-clip could extend fully around the axis, with the two ends of the c-clip at the notch abutting one another). The c-clip could extend between 180 and 359 degrees, for example (for example between 270 and 345 degrees, or more specifically between 300 and 330 degrees) around the longitudinal axis of the syringe carrier in the circumferential direction. A flange 146 extends towards the longitudinal axis from the c-clip. The flange 146 comprises an optional notch 230 so that the c-clip can more easily expand.

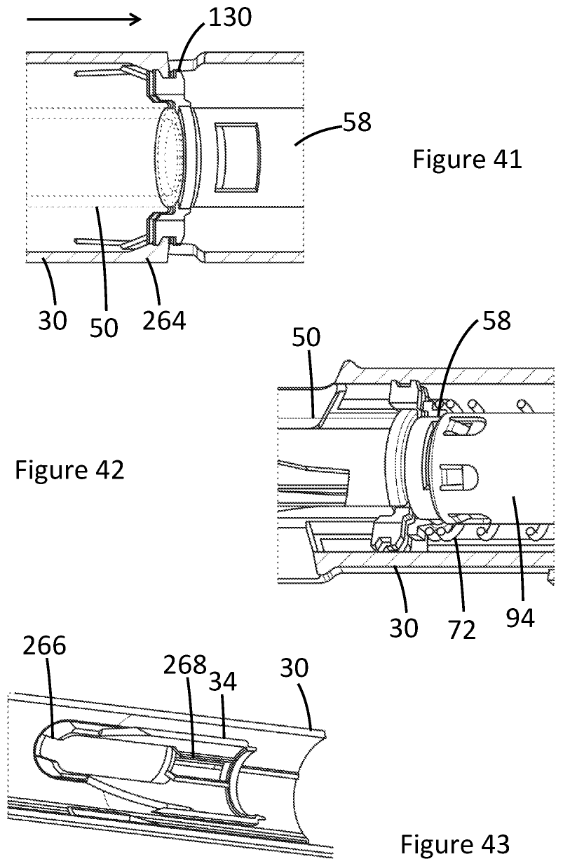

In this example, the syringe carrier 130 is first inserted into the distal end of a housing of a medicament delivery device (the direction of insertion is shown in FIG. 41 by an arrow). The arms 250 of the syringe carrier engage the housing. Variously shaped features such as arms, recesses, cut-outs and protrusions could be provided on the housing to engage the syringe carrier, but in this case proximally extending flexible arms 264 of the housing 30 are provided, with the distal end of the arms 264 attached to the housing and the proximal end of the arms 264 free to move radially relative to the housing, and with radially inwardly extending protrusions on the proximal end of the arms 264 to engage the arms 250 of the syringe carrier. The distally facing surfaces of the radially inwardly extending protrusions are preferably angled towards the axis to reduce the friction when inserting the syringe carrier.

Once the syringe carrier is inserted into the housing, with the arms of the syringe carrier engaged with the housing, the syringe can be inserted into the syringe carrier, resulting in the arrangement shown in FIG. 41.

A feature of the housing 30 (in this case the arms 264 with inwardly extending protrusions as just described, see FIG. 41) can be used to keep the syringe carrier in a particular place during assembly. In this example, the point at which the syringe carrier is released can be controlled by initially restricting movement of the arms (and hence the protrusions) away from the axis during assembly, thereby fixing the position of the housing relative to the syringe carrier. When this restriction is subsequently removed, the syringe carrier can then be disengaged from the protrusions, thereby allowing the syringe carrier to move relative to the housing.

From the position shown in FIG. 41, the syringe and the syringe carrier are moved in the proximal direction relative to the housing to a final position as shown in FIG. 42. For context, several other example medicament delivery device features are shown in FIG. 42; these will not be described in detail as they have already been described in other parts of the application, for example with reference to FIGS. 1 and 44.

In the final position as shown in FIG. 42, the syringe housing could be attached to the housing, for example by a snap fit or friction fit, to help the syringe carrier support the syringe. In some medicament delivery devices, it may prove necessary to include one or more cut-outs or supporting features on certain interior structures of the housing (for example the cut-out 266 of the window rim 252 as shown in FIG. 43 and the pathway 268 described by ribs on the inner housing 34—these ribs could also help support the syringe), to create a pathway for the arms (carrier structure) on the syringe carrier.

The examples herein focus on autoinjectors, but the examples described herein could be implemented in other medicament delivery devices more generally, such as in pen injectors. Some of the examples herein focus on 1 ml syringes, but the designs described herein could also be used on other volumes and other types of medicament container, for example a syringe without an attached needle rather than a syringe with an attached needle. An example of an autoinjector 10 that could comprise the syringe carriers described herein is shown in FIG. 44. The example autoinjector extends along an axis 12 in an axial direction 13 between a distal end 15 and a proximal end 14, with a radial direction 17 and a circumferential direction 16 also depicted for reference. A housing (or body) 30 and a cap 90 of the autoinjector can be seen, along with an optional window 32 in the body. The autoinjector can house a syringe. The autoinjector typically includes features such as a powerpack and a needle guard inside the housing. The shape of the housing and of the cap could be varied from those shown in the example—for example, the housing could be triangular in cross section perpendicular to the axis rather than circular, could be an irregularly-shaped tube rather than a cylinder, and/or the housing could be two or more components rather than a single component. The autoinjector shown does not have an activation button, though one could be provided (i.e. a three-step autoinjector rather than a two-step autoinjector).

FIGS. 45 and 46 show an example of a syringe for reference. This particular syringe 50 comprises a medicament holder (medicament container) 52, a needle 54, a stopper 56, a rigid needle shield (RNS) 58, a flexible needle shield (FNS) 60, a flange 62 and a shoulder 64. The syringe extends from a proximal end 14 to a distal end 15. The medicament holder 52 is tubular (specifically cylindrical in this example), with the flange 62 at the distal end of the medicament holder 52 and the needle 54 at the proximal end of the medicament holder 52. The stopper 56 is in the medicament holder 52. The flexible needle shield 60 extends around the needle 54, and the rigid needle shield 58 extends around the flexible needle shield. The shoulder 64 is the proximal end of the medicament holder 52. One particular example syringe is described here, but other syringes could be used. For example, a needle 54 is included in examples described herein, but other medicament delivery members such as jet injectors could alternatively be used, or the needle could be provided separately rather than as an integral part of the syringe. A needle shield comprising a rigid needle shield 58 and a flexible needle shield 60 is included in examples described herein, but the examples described herein could be used with needle shields without a flexible needle shield or even entirely without a needle shield, although the examples described herein can be particularly beneficial when used with syringes with an RNS. The syringe could be various sizes, including but not limited to 1 ml and 2.25 ml.

Example mechanical powerpacks are described herein (for example the powerpacks shown in FIGS. 1 and 19), but other types of powerpack could be used instead, for example an electrically powered powerpack or a gas-powered powerpack. An example of a device in which syringe carriers as described herein could be used is provided in WO2011/123024, which is hereby incorporated by reference.

A base 132 (base section) is described herein. Typically, the base is depicted as the distal portion of the syringe carrier, but could alternatively be spaced apart in the axial direction from the distal end of the syringe carrier.

Arms 134 are described herein. The examples herein use two arms, although in the examples with two arms, one, three or more arms could alternatively be provided. Similarly, other features that are provided (arms, protrusions, cut-outs, recesses and the like) can generally be provided in a quantity different to the specific number described in the examples given. In general, the arms could be replaced by another connection section such as the flexible sleeve shown in FIGS. 21 and 22.

Various rings 136 are described herein. Some of the example rings comprise one or more hinges. The ring (or more specifically the hinges of the ring) would typically be biased inwards so that the ring automatically contracts after the RNS has passed, although this is not essential and the ring could be contracted in a subsequent step (manually or with a machine or by another part of the medicament delivery device during assembly).

The syringe carriers described herein generally have a fixed circumference—that is, the actual material of the ring (or the c-clip) does not expand, but the ring (or c-clip) is flexible so that the shape of the ring can change. More generally, the flexibility of the ring (or c-clip) is such that the shape of the ring (or c-clip) can change to change the shape (and in some cases also the area) of the space inside the ring (or c-clip)(when looking at a cross-section of the ring (or c-clip) in a plane perpendicular to the longitudinal axis).

Flanges 146 are described herein. These could vary in shape, particularly depending on the shoulder 64 and the RNS 58 of the syringe 50, but could have a proximal face facing in the proximal direction and a distal face facing at an angle from the distal direction (i.e. sloped) as shown in FIG. 40. A priority with the flange is that it is configured to fit in the gap between the shoulder 64 and the RNS 58 of the syringe 50. When no flange is provided, another feature of the syringe carrier would typically need to extend into the gap between the shoulder 64 and the RNS 58 (or at least to extend so that it supports the shoulder 64) to help support the syringe.

Many of the syringe carriers described herein comprise some kind of optional protrusion or rib. For example, a number of the examples, including the syringe carriers shown in FIGS. 2, 6 and 14, comprise two ribs 138, four second ribs 139 and two protrusions 140. The ribs 138 extend in the longitudinal direction, with the proximal end of each rib 138 attached to an arm 134 and the distal end of each rib 138 attached to the base 132. The second ribs 139 extend in the longitudinal direction. A second rib 139 is arranged on each side of each arm 134 in the circumferential direction. The protrusions 140 are attached to the base 132. Another optional feature is a distal flange 148, which is also present in many of the syringe carriers described herein. The flange 148 is attached to the base 132 and extends in the radial direction away from the axis and extends in the circumferential direction around the base. These ribs, protrusions and flanges can provide various advantages, including helping to align the syringe carrier relative to other features during assembly, maintaining rigidity of the syringe carrier, and/or fixing the position of the syringe carrier relative to other components of a medicament delivery device in a completed device. Whilst these protrusions 140, ribs 138, 139 and flanges 148 are depicted as having a particular shape, these shapes could be varied depending on factors such as the desired rigidity and on the shape of other components within a particular design of medicament delivery device. Various example rings are provided with specific numbers of segments 142 and hinges 144. In general, the numbers of segments and hinges can be varied from the numbers shown.

In the present disclosure, when the term “distal direction” is used, this refers to the direction pointing away from the dose delivery site during use of the medicament delivery device. When the term “distal part/end” is used, this refers to the part/end of the delivery device, or the parts/ends of the members thereof, which under use of the medicament delivery device is/are located furthest away from the dose delivery site. Correspondingly, when the term “proximal direction” is used, this refers to the direction pointing towards the dose delivery site during use of the medicament delivery device. When the term “proximal part/end” is used, this refers to the part/end of the delivery device, or the parts/ends of the members thereof, which under use of the medicament delivery device is/are located closest to the dose delivery site.

Further, the terms “longitudinal”, “longitudinally”, “axially” and “axial” refer to a direction extending from the proximal end to the distal end and along the device or components thereof, typically in the direction of the longest extension of the device and/or component. The circumferential direction describes a direction extending around the axis, so around the circumference of a device or component, and the radial direction extends perpendicular to the axis.

Similarly, the terms “transverse”, “transversal” and “transversally” refer to a direction generally perpendicular to the longitudinal direction.

Generally, all terms used in the claims are to be interpreted according to their ordinary meaning in the technical field, unless explicitly defined otherwise herein. All references to a/an/the element, apparatus, member, component, means, etc. are to be interpreted openly as referring to at least one instance of the element, apparatus, member component, means, etc., unless explicitly stated otherwise.

Various modifications to the embodiments described are possible and will occur to those skilled in the art without departing from the present disclosure which is defined by the following claims.

Some aspects of the present disclosure are summarised in the following clauses.

1. A syringe carrier (130) for a medicament delivery device (10), wherein the syringe carrier (130) is tubular and extends along a longitudinal axis from a proximal end to a distal end, the syringe carrier (130) comprising
a base section (132) at the distal end of the syringe carrier (130), the base section (132) having a fixed circumference,
at least one arm (134) extending towards the proximal end of the syringe carrier (130) from the base section (132), and
an expandable ring (136) attached to the proximal end of the at least one arm (134), wherein the ring (136) extends entirely around the longitudinal axis of the syringe carrier (130) in the circumferential direction relative to the longitudinal axis.

2. The syringe carrier of clause 1, wherein the expandable ring is arranged to expand by breaking.

3. The syringe carrier of clause 2, wherein the expandable ring comprises a weak point at which the expandable ring is designed to break when a syringe is inserted into the syringe carrier.

4. The syringe carrier of clause 1, wherein the expandable ring comprises one or more hinges spaced around the circumference of the expandable ring.

5. The syringe carrier of clause 4 wherein the expandable ring comprises a plurality of segments spaced around the circumference of the expandable ring and a plurality of hinges spaced around the circumference of the expandable ring, and wherein each segment has a hinge on either side in the circumferential direction.

6. The syringe carrier of clause 4 or 5, wherein at least one of the one or more hinges is a collet.

7. The syringe carrier of clause 6, wherein the collet extends entirely around the longitudinal axis of the syringe carrier in the circumferential direction.

8. The syringe carrier of any previous clause, wherein the expandable ring is circular in a cross-section perpendicular to the longitudinal axis.

9. The syringe carrier of any previous clause, wherein the expandable ring is arranged to expand by increasing the diameter of the ring.

10. A syringe carrier for a medicament delivery device, wherein the syringe carrier is tubular and extends along a longitudinal axis from a proximal end to a distal end, the syringe carrier comprising
a base section at the distal end of the syringe carrier,
a connection section extending towards the proximal end of the syringe carrier from the base section, and
a non-circular ring attached to the proximal end of the connection section, wherein the ring extends entirely around the longitudinal axis of the syringe carrier in the circumferential direction.

11. The syringe carrier of clause 10, wherein the connection section is at least one flexible arm.

12. The syringe carrier of clause 10, wherein the connection section is a flexible sleeve.

13. The syringe carrier of any of clauses 10 to 12, wherein the ring is oval.

14. The syringe carrier of any of clauses 10 to 12, wherein the ring has a plurality of sections, and a first subset of the sections are further from the longitudinal axis than a second subset of the sections.

15. The syringe carrier of any of clauses 10 to 12, wherein the ring has a plurality of sections, and at least one of the sections has a radius of curvature smaller than the average radius of curvature of the overall ring.

16. The syringe carrier of any previous clause, wherein the at least one arm extends in the longitudinal direction from a proximal end to a distal end, and wherein the expandable ring is attached to the proximal end of each of the two arms.

17. The syringe carrier of any previous clause, wherein the at least one arm is flexible.

18. The syringe carrier of any previous clause, wherein the at least one arm comprises a slot extending in the longitudinal direction from the proximal end of the arm.

19. The syringe carrier of any previous clause, wherein the at least one arm is attached to the part of the ring that is furthest from the longitudinal axis.

20. The syringe carrier of any previous clause, wherein the base extends entirely around the longitudinal axis of the syringe carrier in the circumferential direction.

21. The syringe carrier of any previous clause, wherein the base is circular in a cross-section perpendicular to the longitudinal axis.

22. The syringe carrier of any previous clause, wherein the proximal end of the syringe carrier is the end that would be closest to the medicament delivery member end when the syringe carrier is assembled in a medicament delivery device.

23. The syringe carrier of any previous clause, wherein the ring is the proximal-most part of the syringe carrier.

24. The syringe carrier of any previous clause, wherein the ring comprises a flange extending towards the longitudinal axis from the ring.

25. A syringe carrier extending along a longitudinal axis from a proximal end to a distal end, the syringe carrier comprising:
a support portion configured to support a shoulder of a syringe of a medicament delivery device, wherein the support portion comprises an expandable base extending around the longitudinal axis in a circumferential direction and a flange extending inwards from the base; and
an attachment portion comprising an arm configured to secure the syringe carrier inside said medicament delivery device, wherein the arm is attached directly to the base.

26. The syringe carrier of clause 25, wherein the base is an expandable ring that extends entirely around the longitudinal axis of the syringe carrier in the circumferential direction.

27. The syringe carrier of clause 26, wherein the expandable ring comprises a plurality of segments around the circumference of the expandable ring and a plurality of hinges around the circumference of the expandable ring, and wherein each segment is attached to a hinge on either side in the circumferential direction.

28. The syringe carrier of clause 25 or 26, wherein the ring is circular.

29. The syringe carrier of clause 25, wherein the base is an expandable c-clip.

30. The syringe carrier of clause 29, wherein the c-clip extends only part of the way around the longitudinal axis of the syringe carrier in the circumferential direction.

31. The syringe carrier of clause 30, wherein the c-clip extends between 180 and 359 degrees, or preferably between 270 and 345 degrees, or most preferably between 300 and 330 degrees around the longitudinal axis of the syringe carrier in the circumferential direction.

32. The syringe carrier of any of clauses 25 to 31, wherein the arm is flexible.

33. The syringe carrier of any of clauses 25 to 32, wherein the arm is attached to an outer surface of the base.

34. The syringe carrier of any of clauses 25 to 33, wherein the arm extends in a distal direction from the support portion.

35. The syringe carrier of any of clauses 25 to 34, wherein the arm comprises a surface facing towards the proximal end, the surface being configured to engage a distally facing surface of a medicament delivery device during medicament delivery device assembly.

36. The syringe carrier of any of clauses 25 to 33, wherein the arm comprises a radially extending protrusion or recess, wherein the protrusion or recess is configured to engage a radially extending feature of a medicament delivery device during medicament delivery device assembly.

37. The syringe carrier of any of clauses 25 to 36, wherein the syringe carrier consists of the support portion and the attachment portion.

38. The syringe carrier of any of clauses 25 to 37, wherein the arm is configured to engage a first attachment of said medicament delivery device during medicament delivery device assembly and a second attachment of said medicament delivery device in a completed medicament delivery device.

39. The syringe carrier of any of clauses 1 to 38, wherein the ring has a fixed circumference.

40. A syringe carrier, the syringe carrier comprising an expandable ring and an attachment portion,
wherein the attachment portion is attached to a segment of the expandable ring, and
wherein the attachment portion comprises an arm configured to engage a first medicament delivery device attachment point during assembly and to subsequently engage a second medicament delivery device attachment point in a completed medicament delivery device.

41. A medicament delivery device sub-assembly comprising a housing, a syringe carrier according to any of clauses 25 to 40 and a syringe,
wherein the housing is tubular and extends from a proximal end to a distal end,
wherein the housing comprises a first attachment point configured to engage the arm of the syringe carrier,
wherein the housing comprises a second attachment point configured to engage the arm of the syringe carrier, and
wherein the second attachment point is closer to the proximal end of the housing than the second attachment point.

42. The medicament delivery device sub-assembly of clause 41, wherein the first attachment point is a rim of a window of the housing.

43. The medicament delivery device sub-assembly of clause 41 or 42, wherein the second attachment point is on an inner portion of the housing.

44. The medicament delivery device sub-assembly of any of clauses 41 to 43, wherein the first attachment point is configured to releasably engage the arm of the syringe carrier, and wherein the second attachment point is configured to permanently engage the arm of the syringe carrier.

45. A medicament delivery device comprising the syringe carrier of any of clauses 1 to 40 or the medicament delivery device sub-assembly of any of clauses 41 to 44.

A medicament delivery device according to clause 45, in which the medicament delivery device is an autoinjector.

46. A method of assembly of a medicament delivery device, the method comprising carrying out the following steps in the following order:
providing a medicament delivery device housing, the housing extending from a proximal end to a distal end;
inserting a syringe carrier according to one of clauses 1 to 40 into the distal end of the housing and moving the syringe carrier in the proximal direction relative to the housing to a first position;
inserting a syringe into the syringe carrier; and
moving the syringe carrier and the syringe together in the proximal direction relative to the housing to a second position.

47. The method of clause 46, wherein the syringe carrier surrounds a smaller area in a plane perpendicular to the longitudinal axis in the second position than in the first position.

48. A method of assembling a medicament delivery device comprising the steps of attaching a syringe to a syringe carrier according to any of clauses 1 to 40 and of subsequently inserting the syringe and the syringe carrier into a housing.

49. A method of assembling a medicament delivery device, comprising the steps of providing a syringe carrier and subsequently inserting a syringe into the syringe carrier, wherein inserting the syringe into the syringe carrier breaks a ring of the syringe carrier.

1-15. (canceled)

16. A syringe carrier for a medicament delivery device, wherein the syringe carrier is tubular and extends along a longitudinal axis from a proximal end to a distal end, the syringe carrier comprising
a base section at the distal end of the syringe carrier, the base section having a fixed circumference,
at least one arm extending towards the proximal end of the syringe carrier from the base section, and
an expandable ring attached to the proximal end of the at least one arm, wherein the ring extends entirely around the longitudinal axis of the syringe carrier in the circumferential direction relative to the longitudinal axis.

17. The syringe carrier of claim 16, wherein the expandable ring is arranged to expand by breaking.

18. The syringe carrier of claim 16, wherein the expandable ring comprises one or more hinges spaced around the circumference of the expandable ring.

19. The syringe carrier of claim 18 wherein the expandable ring comprises a plurality of segments spaced around the circumference of the expandable ring and a plurality of hinges spaced around the circumference of the expandable ring, and wherein each segment has a hinge on either side in the circumferential direction.

20. The syringe carrier of claim 18, wherein at least one of the one or more hinges is a collet.

21. A syringe carrier for a medicament delivery device, wherein the syringe carrier is tubular and extends along a longitudinal axis from a proximal end to a distal end, the syringe carrier comprising
a base section at the distal end of the syringe carrier,
a connection section extending towards the proximal end of the syringe carrier from the base section, and
a non-circular ring attached to the proximal end of the connection section, wherein the ring extends entirely around the longitudinal axis of the syringe carrier in the circumferential direction.

22. The syringe carrier of claim 21, wherein the connection section is at least one flexible arm.

23. The syringe carrier of claim 21, wherein the connection section is a flexible sleeve.

24. The syringe carrier of claim 21, wherein the ring is oval.

25. The syringe carrier of claim 21, wherein the ring has a plurality of sections, and a first subset of the sections are further from the longitudinal axis than a second subset of the sections.

26. A syringe carrier extending along a longitudinal axis from a proximal end to a distal end, the syringe carrier comprising:
a support portion configured to support a shoulder of a syringe of a medicament delivery device, wherein the support portion comprises an expandable base extending around the longitudinal axis in a circumferential direction and a flange extending inwards from the base; and
an attachment portion comprising an arm configured to secure the syringe carrier inside said medicament delivery device, wherein the arm is attached directly to the base.

27. The syringe carrier of claim 26, wherein the base is an expandable ring that extends entirely around the longitudinal axis of the syringe carrier in the circumferential direction.

28. The syringe carrier of claim 27, wherein the expandable ring comprises a plurality of segments around the circumference of the expandable ring and a plurality of hinges around the circumference of the expandable ring, and wherein each segment is attached to a hinge on either side in the circumferential direction.

29. The syringe carrier of claim 26, wherein the base is an expandable c-clip.

30. The syringe carrier of claim 26, wherein the ring is circular.

31. The syringe carrier of claim 29, wherein the c-clip extends only part of the way around the longitudinal axis of the syringe carrier in the circumferential direction.

32. The syringe carrier of claim 29, wherein the c-clip extends between 180 and 359 degrees around the longitudinal axis of the syringe carrier in the circumferential direction.

33. The syringe carrier of any of claim 26, wherein the arm is flexible.

34. The syringe carrier of any of claim 26, wherein the arm is attached to an outer surface of the base and extends in a distal direction from the support portion.

35. A medicament delivery device sub-assembly comprising a housing, a syringe carrier according to claim 26 and a syringe,
wherein the housing is tubular and extends from a proximal end to a distal end,
wherein the housing comprises a first attachment point configured to engage the arm of the syringe carrier,
wherein the housing comprises a second attachment point configured to engage the arm of the syringe carrier, and
wherein the second attachment point is closer to the proximal end of the housing than the second attachment point.
